(19) United States
(12) Patent Application Publication
(10) Pub. No.: US 2018/0303363 A1
(43) Pub. Date: Oct. 25, 2018

(54) METHOD FOR MONITORING AND EVALUATING CARDIAC ANOMALIES

(71) Applicant: NATIONAL CARDIAC, INC., Escondido, CA (US)

(72) Inventors: Paul M. DiPerna, Escondido, CA (US); Freeman H. Rose, JR., Del Mar, CA (US)

(21) Appl. No.: 15/492,775

(22) Filed: Apr. 20, 2017

(51) Int. Cl. A61B 5/04; A61B 5/0452; A61B 5/00

(52) U.S. Cl. CPC A61B 5/04012; A61B 5/0452; A61B 2560/0214; A61B 2560/0247; A61B 5/746

(57) ABSTRACT

The present invention pertains to methods for monitoring and evaluating a patient's heart muscle function. For the present invention this requires simultaneously detecting both cardiac signals (i.e. an EKG) and perturbation signals caused by the environmental and physical factors that are influencing the patient. In particular, when a cardiac signal is not compliant with a predetermined cardio-profile, an anomaly results. The resultant anomaly, and the perturbation causing the anomaly are then evaluated to determine whether an appropriate action is required.

### FIELD OF THE INVENTION

The present invention pertains generally to methods for monitoring and evaluating a patient's heart muscle function. More particularly, the present invention pertains to methods for evaluating anomalies in the cardiac signals of a patient that are caused by an external influence (i.e. an environmental or a physical perturbation). The present invention is particularly, but not exclusively, useful as a method for determining when a cardiac signal anomaly is not compliant with a predetermined cardio-profile and requires an appropriate action.

### BACKGROUND OF THE INVENTION

It is well known that the heart muscle function of a patient (user) can be affected by either external (i.e. extracorporeal) influences or internal (i.e. systemic) influences. Whereas internal influences are typically chronic in nature, external influences are typically acute. Also, external influences are typically the result of either physical or environmental stimuli which are experienced by a person within a relatively brief period of time. For both cases however, whenever it is determined that a patient's heart muscle function may be at risk, an ability to monitor and evaluate the situation can be of considerable importance for the patient. In particular, it will be important to not only detect when there is an anomaly in the heart muscle function, but to also determine what is the cause of the anomaly, its severity, and the affect it can have on a patient's health and wellbeing.

In accordance with accepted clinical practices, the heart muscle function of any patient can be recorded as a waveform, using sensors such as an electrocardiograph (EKG). Moreover, because they are individually unique, the EKG waveform for each patient will exhibit measurable parameters that can be identified and evaluated by a trained clinician. In particular, such parameters can be identified to establish an acceptable operating envelope (i.e. cardio-profile) for the heart muscle function of the particular patient (user).

Using the cardio-profile as a start point or benchmark, perturbations to the EKG waveform that cause a measurable parameter to become non-compliant with the cardio-profile can be identified for further consideration. For this purpose, the nature, extent, and severity of a perturbation may be of particular importance. Specifically, the effect a perturbation has in creating an anomaly of the heart muscle function will be useful for determining whether immediate medical attention is required, routine medical attention will suffice, or no action needs to be taken.

In light of the above, an object of the present invention is to provide a methodology for monitoring and evaluating cardiac anomalies, whereby a cardio-profile is established to identify cardiac anomalies resulting from internally or externally caused perturbations that can be evaluated for requisite medical attention. Another object of the present invention is to provide a methodology for monitoring and evaluating cardiac anomalies that is easy to use, is effective for its intended purpose and is comparatively cost effective to implement.

### SUMMARY OF THE INVENTION

In accordance with the present invention, a methodology for monitoring heart muscle activity of a patient (user) involves detecting a cardiac anomaly relative to the patient's normal cardiac waveform or a desired target waveform indicated by a physician. The cardiac anomaly is then evaluated for its response to a concurrent perturbation of the cardiac waveform. For the present invention, this requires employing three different types of sensors. These are: a cardiac sensor for recording the actual, real time, cardiac waveform of the patient; at least one perturbation sensor for simultaneously detecting external and/or internal factors influencing the cardiac waveform in real time; and system sensors that determine whether the cardiac and perturbation sensor(s) is(are) operational.

As a first step in the methodology of the present invention, a cardio-profile is established. For the present invention, this cardio-profile may be either patient-specific, or it may be predetermined. In detail, the creation of a cardio-profile requires selecting pre-identified measurable parameters from the patient's cardiac waveform that can be subsequently monitored on a continuing, or predetermined periodic basis. Further, the cardio-profile establishes acceptable ranges for variations of each parameter in the cardiac waveform that is being monitored.

For a typical operation of the present invention, the parameters selected for monitoring will generally be either temporal or dimensional measurements. For example, the parameters for dimensional measurements will typically include waveform shape characteristics and amplitudes within the waveform. On the other hand, temporal measurements will typically include the repetition rate of heart function cycles within the waveform, variability of the waveform shape, discontinuities in the waveform, and variability in beat to beat timing. Collectively, such parameters and the acceptable ranges for variations of these parameters constitute the cardio-profile. Once the cardio-profile has been established it can be input to a computer.

The cardiac sensor for the present invention will typically be of a type that is well-known in the pertinent art, and is capable of recording the cardiac waveform of the patient's heart muscle function, such as an electrocardiograph (EKG). Preferably, recordation of the waveform is accomplished in real time on a continuing basis. As envisioned for the present invention the cardiac sensor will be conveniently located on the body of the patient (user) and, if needed, it can be implanted. In any event, the cardiac signal that is detected by the cardiac sensor will be provided as a direct, real time input to the computer.

As noted above, the perturbation sensors are used for the present invention to detect perturbations of the heart muscle waveform that are caused by either external or internal influences. These perturbations can be further categorized according to the nature of the influence into either environmental perturbations or physical perturbations. For example, the environmental perturbations will be typically caused by local weather conditions and other factors such as electromagnetic radiations, radioactivity, time of day, climatic considerations, and altitude. On the other hand, physical perturbations will result from factors such as stress, trauma, disease, extrinsic exercise/activity level, sleep patterns, and body contacts. Recall, that the cardiac sensor and the perturbation sensors detect contemporaneous signals simultaneously. Moreover, like the cardiac signal from the cardiac sensor, the perturbation signals from the perturbation sensor are directly input to the computer.

For an operation of the present invention, the cardiac sensor is used to detect cardiac signals that are generated by the heart muscle of the patient (user). Typically, the cardiac signals will be waveforms that are recorded by an EKG, and they will include a continuous sequence of heart muscle function cycles. At the same time that the cardiac signal is being monitored and recorded, an array of perturbation sensors also monitor the environment and physical status of the patient (user).

In the course of events, whenever an external/internal influence that is detected by a perturbation sensor causes an anomaly to simultaneously occur in the cardiac signal, both the cause/source of the perturbation and the nature/extent of the anomaly on the cardiac signal are comparatively evaluated. More specifically, as noted above, when an anomaly is created in the cardiac signal that does not comply with the predetermined cardio-profile, such an evaluation is initiated in the computer. The result of this comparative evaluation is a determination (i.e. a report) as to whether a responsive medical action is required.

An additional feature of the present invention is the optional incorporation of a system sensor with the perturbation sensor. When incorporated, the system sensor will function to monitor the respective operational status of the cardiac sensor and the perturbation sensor. A purpose here is to detect perturbations in the system that may be caused by the patient (user). Also, the system sensor can be used to monitor the system operation for regulatory compliance, and to identify and address maintenance considerations, such as battery charge and operational readiness requirements.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a functional schematic of operational tasks required for the methodology of the present invention;

FIG. 2 is an exemplary cardiac waveform of a patient (user) as would be recorded by an Electrocardiograph;

FIG. 3 is a representative heart function cycle taken from the cardiac waveform shown in FIG. 2;

FIG. 4 is a presentation of amplitude and time based parameters selected from the heart function cycle shown in FIG. 3 for inclusion in a cardio-profile in accordance with the present invention; and

FIG. 5 is a logic flow chart of the sequential tasks and functions required for an operation of the methodology of the present invention.

### DESCRIPTION OF THE PREFERRED EMBODIMENTS

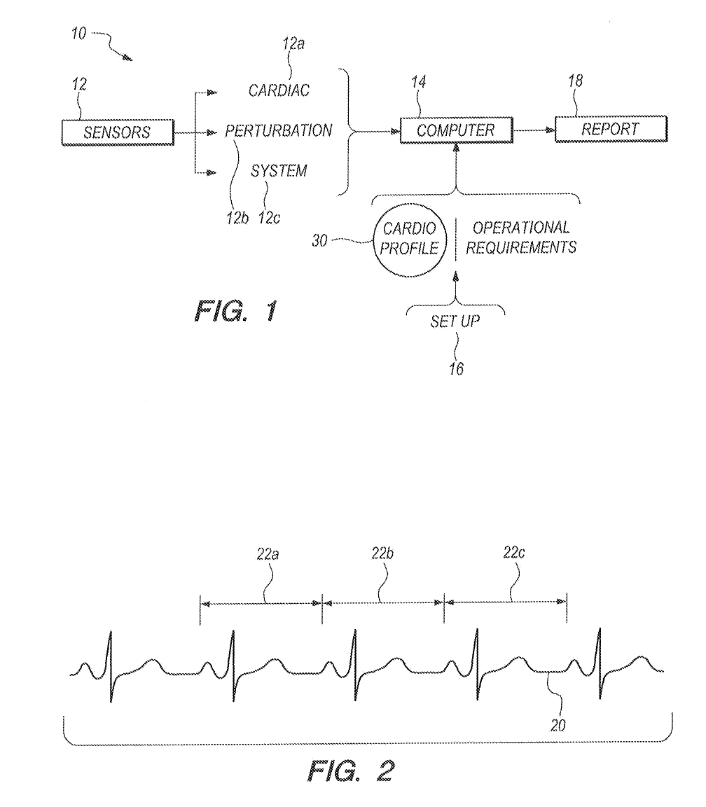

Referring initially to FIG. 1, the various operational tasks that are required for implementing the methodology of the present invention are collectively designated 10. In FIG. 1 they are shown schematically in their interactive relationship with each other. In overview, the methodology 10 requires the use of several different types of sensors 12 which are each directly or indirectly connected with a patient/user (not shown). Data that is collected by the sensors 12 are electronically transmitted to a computer 14 for evaluation. For an operation of the methodology 10, the task 16 shown in FIG. 1 indicates that it is first necessary to set up (i.e. pre-program) the computer 14.

As envisioned for the present invention, the sensors 12 are essentially of three different types. In general these sensors are: a cardiac sensor 12a for recording the actual, real time, cardiac activity of the patient; at least one perturbation sensor 12b for simultaneously detecting external and/or internal factors that can influence the cardiac activity in real time; and system sensors 12c that determine whether the computer 14 and the other sensors 12 are operational. The condition of all sensors 12, and an evaluation by the computer 14 of data collected from the sensors 12 is then compiled by the computer 14 and presented as a report 18.

The primary purpose for the methodology 10 of the present invention is to monitor the waveform 20 of a patient's heart muscle function. With this in mind, the waveform 20 shown in FIG. 2 is typical. More specifically, however, the methodology 10 is provided to monitor the waveform 20 for variations that may be indicative of an adverse effect on the patient's health and wellbeing. To do this, the methodology 10 is primarily concerned with sequentially monitoring individual heart function cycles 22 in the waveform 20. In FIG. 2, the heart function cycles 22a, 22b and 22c of waveform 20 are only exemplary. Importantly, for a normal heart function, all cycles 22 are essentially similar.

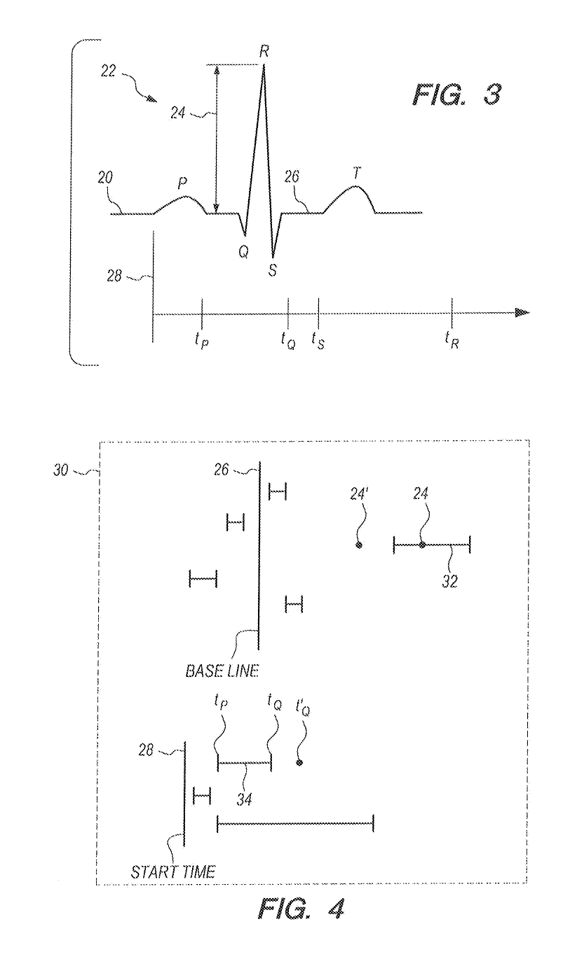

As shown in FIG. 3 a single heart function cycle 22 includes universally recognized points (P, Q, R, S, and T) in the waveform 20, which are characteristic of the particular heart function cycle 22. For purposes of this disclosure, the characteristics of a heart function cycle 22 are recognized as having various measurable parameters that collectively exemplify the efficacy of the heart muscle function.

In FIG. 3 it will be appreciated that both dimensional and temporal parameters of a heart function cycle 22 can be measured by the cardiac sensor 12a. For example, the amplitude 24 of R is a dimensional parameter that can be measured relative to a base line 26. Likewise, the amplitudes of P, Q, S and T have respective amplitudes that are similarly measurable from the same base line 26. Further, the occurrence of P, Q, R, S and T can also be measured relative to a start time 28 as a temporal parameter. With all of this in mind, it will be appreciated that heart function cycles 22 are patient-specific. Moreover, for each patient, P, Q, R, S and T will have a respective range of acceptable amplitudes, and each will have an acceptable range for the time during which they occur in the heart function cycle 22. Other parameters, such as the temporal spacing between subsequent QRS cycles, or the variability of that spacing, may also apply. For purposes of the present invention, these amplitude ranges (dimensional) and occurrence ranges (temporal) within a heart function cycle 22 are collectively referred to here as a cardio-profile 30.

Referring now to FIG. 4, a cardio-profile 30 is shown as a collection of the acceptable ranges that are clinically established for respective dimensional and temporal parameters of a heart function cycle 22. As implied above, when all measurable parameters remain within their respective acceptable ranges, the heart muscle function is considered normal. However, when any measurable parameter falls outside its acceptable range an anomaly occurs which requires further evaluation. In FIG. 4 the amplitude 24 of R and the time interval between P and Q in the cardio-profile 30 are used as examples for purposes of disclosure.

In FIG. 4 a range 32 for the amplitude 24 of R is dimensionally established relative to the base line 26. In this example, the range 32 is considered acceptable for variations of the amplitude 24. Accordingly, an amplitude 24′ which is outside the range 32 when detected by the cardiac sensor 12a would be considered to be an anomaly. Similarly, from a temporal perspective, the range 34 is considered to be an acceptable time interval between the occurrence of P, at time t_{P}, and the occurrence of Q, at time t_{Q}. In this example, an occurrence of Q at the time t_{Q}′ when it is detected by the cardiac sensor 12a outside the range 32, would be considered an anomaly. Likewise, the amplitudes for P, Q, S and T, as well as the times t_{P}, t_{R}, t_{S}, and t_{T} in the heart function cycle 22 are also similarly evaluated in comparison with the cardio-profile 30 to identify respective anomalies. In the event, an analysis and evaluation of a heart muscle function cycle 22 as disclosed above can be used to identify anomalies in waveform shape characteristics, amplitudes within the waveform, the repetition rate of heart function cycles in the waveform, variability of the waveform shape, and discontinuities in the waveform.

For the present invention, the detection of anomalies in a heart muscle function cycle 22 does not end the inquiry. Instead, in accordance with the methodology 10 of the present invention, the extent, severity, and cause of an anomaly are evaluated relative to a simultaneously occurring perturbation. To do this, perturbation sensors 12b are appropriately positioned relative to the patient (user). For purposes of the present invention perturbation signals detected by the perturbation sensors 12b will typically include both environmental perturbations and physical perturbations. In particular, the environmental perturbations will typically involve local weather conditions, electromagnetic radiations, radioactivity, time of day, climatic considerations, and altitude. On the other hand, physical perturbations will typically involve stress, trauma, disease, extrinsic exercise/activity level, sleep patterns, and body contacts.

As an additional feature of the present invention a system sensor 12c can be incorporated with the perturbation sensor 12b for monitoring a respective operational status of the cardiac sensor 12a and the perturbation sensor 12b. Specifically, the system sensor 12c will be incorporated to detect system perturbations that may be caused by the patient (user). In particular these perturbations will relate to compliance and system maintenance considerations, and will include considerations for such matters as battery charge and operational readiness requirements.

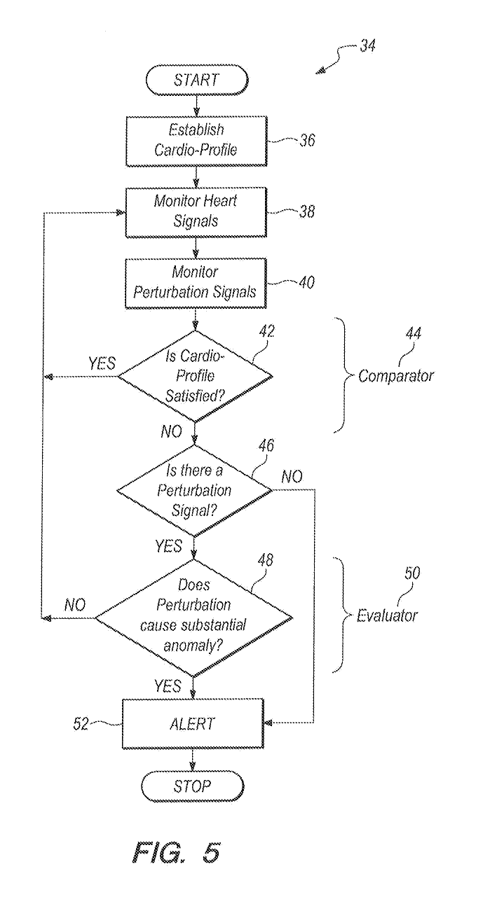

An operation for the methodology 10 of the present invention is presented by the logic flow chart which is generally designated 34 in FIG. 5. As indicated by the block 36 of chart 34 in FIG. 5, it is important that a cardio-profile 30 be initially established, or selected, for the particular patient (user). In FIG. 1, this requirement is shown as part of task 16 for setting up the computer 14. Also, during this initial setup, the operational requirements to be monitored by the systems sensor 12c are also input to the computer 14. Then, once the cardio-profile 30 is established, and the sensors 12 have all been properly positioned and located, block 38 indicates that the cardiac sensor 12a is activated to monitor the cardiac waveform 20. Simultaneously, block 40 indicates that the perturbation sensor(s) 12b are also activated to monitor for environmental and physical perturbations. Importantly, as noted above, perturbations sensors 12b operate concurrently with the cardiac sensor 12a and, thus, they provide for a comparison of the data that is received simultaneously by the sensors 12a and 12b.

Inquiry block 42 of chart 34 indicates that the cardiac signals detected by cardiac sensor 12a are compared directly with the cardio-profile 30. Preferably, this comparison is made by a comparator 44 that is incorporated into the computer 14. If this comparison determines that the cardiac signal is compliant with the cardio-profile 30, chart 34 shows that the methodology 10 requires continued monitoring by the cardiac sensor 12a and the perturbation sensors 12b. On the other hand, if the comparator 44 determines the cardiac signal that is detected by the cardiac sensor 12a is not compliant with the cardio-profile 30, the methodology 10 determines that an anomaly has occurred and the methodology 10 continues to the inquiry block 46.

At the inquiry block 46 the methodology 10 questions whether a perturbation signal has been received from a perturbation sensor 12b. If there is such a perturbation signal, the methodology 10 proceeds to inquiry block 48 and makes further inquiry into whether the anomaly is substantial. In particular, the determination of substantiality is made by an evaluator 50 that is incorporated into the computer 14 and it is based on an overall evaluation of the effect a particular perturbation has had on the cardiac waveform 20. In the event the determination of substantiality is that the perturbation was minimal, and likely had no long term adverse effect on the patient (user), the methodology returns to block 38. The cardiac sensor 12a and the perturbation sensor 12b then continue their respective monitoring activity.

It is to be noted that in accordance with the methodology 10 presented in chart 34, an alert signal 52 is generated under the following three scenarios. First, when there is no cardiac signal from the cardiac sensor 12a that can be compared with the cardio-profile the alert signal 52 is activated (see inquiry block 42). Second, when according to inquiry block 48, the evaluator 50 in computer 14 determines a substantial anomaly has occurred. And third, the alert signal 52 is activated when the inquiry block 46 determines that no perturbation signal is being received from the perturbation sensor 12b (see inquiry block 46). In each of these situations, the methodology 10 requires that some form of assessment is to be made.

While the particular Method for Monitoring and Evaluating Cardiac Anomalies as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A method for monitoring and evaluating cardiac anomalies which comprises the steps of:
establishing a cardio-profile for a patient, wherein the cardio-profile identifies certain measurable parameters of cardiac signals generated by the heart muscle of the patient, and wherein the cardio-profile establishes acceptable ranges for variations in individual parameters of a cardiac signal;
detecting the cardiac signals generated by the heart muscle of the patient;
detecting perturbation signals experienced by the patient, wherein the cardiac signals and the perturbation signals are correspondingly detected simultaneously;
comparing the cardiac signals with the cardio-profile to identify when a perturbation signal causes an anomaly; and
evaluating the perturbation signal causing the anomaly to determine whether the resultant anomaly requires an appropriate action.

2. The method recited in claim 1 wherein the measurable parameters of cardiac signals from the heart muscle are selected from the group consisting of waveform shape characteristics, amplitudes within the waveform, the repetition rate of heart function cycles in the waveform, variability of the waveform shape, discontinuities in the waveform, and variability of the repetition rate.

3. The method recited in claim 1 wherein an anomaly is identified when a perturbation extends the cardiac signal beyond an acceptable range in the cardio-profile.

4. The method recited in claim 1 wherein the perturbation signals include environmental perturbations and physical perturbations, wherein the environmental perturbations are respectively caused by local weather conditions, electromagnetic radiations, radioactivity, time of day, climatic considerations, and altitude, and wherein the physical perturbations are respectively caused by stress, trauma, disease, extrinsic exercise/activity level, sleep patterns, and body contacts.

5. The method recited in claim 1 further comprising the steps of:
transmitting an alert to a remote facility whenever an anomaly requires an active medical response; and
receiving information from a remote facility to update the cardio-profile when needed.

6. The method recited in claim 1 wherein the cardiac sensor and the perturbation sensor are mounted on a same substrate.

7. The method recited in claim 1 wherein the cardiac sensor is implanted in the torso of the patient.

8. The method recited in claim 1 wherein the cardiac sensor is an electrocardiograph, and wherein the perturbation sensor is an array of sensors, wherein each sensor in the array detects a respectively different perturbation.

9. A method for using sensors to evaluate the heart muscle function of a patient which comprises the steps of:
establishing a cardio-profile for the patient, wherein the cardio-profile identifies certain measurable parameters of cardiac signals generated by the heart muscle of the patient, and wherein the cardio-profile establishes acceptable ranges for variations in individual parameters of a cardiac signal;
locating a cardiac sensor on the patient to monitor and record cardiac signals generated by the heart muscle of the patient;
positioning a perturbation sensor on the patient to detect a perturbation signal, wherein the perturbation signal is caused by an external influence experienced by the patient affecting at least one parameter of the cardiac signals detected by the cardiac sensor;
incorporating a system sensor with the perturbation sensor for monitoring a respective operational status of the cardiac sensor and the perturbation sensor, to detect system perturbations caused by patient compliance and system maintenance considerations, to include battery charge and operational readiness requirements;
comparing each cardiac signal with the cardio-profile to identify when a corresponding perturbation signal causes an anomaly;
evaluating the perturbation signal causing the anomaly to determine whether the resultant anomaly requires an appropriate action; and
providing a report in response to indications from the incorporating step and from the evaluating step signifying when appropriate action is required.

10. The method recited in claim 9 wherein the measurable parameters of signals from the heart muscle are selected from the group consisting of waveform shape characteristics, amplitudes within the waveform, the repetition rate of heart function cycles in the waveform, variability of the waveform shape, discontinuities in the waveform, and variability of the repetition rate.

11. The method recited in claim 9 wherein an anomaly is identified when a perturbation signal extends the cardiac signal beyond an acceptable range in the cardio-profile.

12. The method recited in claim 9 wherein the perturbation signals include environmental perturbations and physical perturbations, wherein the perturbation signals include environmentally caused perturbations and physically caused perturbations, wherein the environmental perturbations are respectively caused by local weather conditions, electromagnetic radiations, radioactivity, time of day, climatic considerations, and altitude, and wherein the physical perturbations are respectively caused by stress, trauma, disease, extrinsic exercise/activity level, sleep patterns, and body contacts.

13. The method recited in claim 9 further comprising the steps of:
transmitting an alert to a remote facility whenever an anomaly requires an active medical response; and
receiving information from a remote facility to update the cardio-profile when needed.

14. The method recited in claim 9 wherein the cardiac sensor and the perturbation sensor are mounted on a same substrate.

15. The method recited in claim 9 wherein the cardiac sensor is implanted in the torso of the patient.

16. The method recited in claim 9 wherein the cardiac sensor is an electrocardiograph, and wherein the perturbation sensor is an array of sensors, wherein each sensor in the array detects a respectively different perturbation.

17. A non-transitory, computer-readable medium having executable instructions stored thereon that direct a computer system to perform a process for monitoring and evaluating cardiac anomalies, the medium comprising instructions for:
establishing a cardio-profile for a patient, wherein the cardio-profile identifies certain measurable parameters of cardiac signals generated by the heart muscle of the patient, and wherein the cardio-profile establishes acceptable ranges for variations in individual parameters of the cardiac signal;
recording cardiac signals generated by the heart muscle of the patient;
detecting a perturbation signal, wherein the perturbation signal is caused by an external influence experienced by the patient affecting at least one parameter of the cardiac signals detected by the cardiac sensor;
monitoring an operational status of the cardiac sensor and of the perturbation sensor, to detect system perturbations respectively caused by patient compliance and system maintenance considerations;
comparing recorded cardiac signals with the cardio-profile to identify when a perturbation signal causes an anomaly; and
evaluating the perturbation signal to determine whether the resultant anomaly requires an appropriate action.

18. The medium recited in claim 17 wherein the measurable parameters of signals from the heart muscle are selected from the group consisting of waveform shape characteristics, amplitudes within the waveform, the repetition rate of heart function cycles in the waveform, variability of the waveform shape, discontinuities in the waveform, and variability of the repetition rate.

19. The medium recited in claim 18 further comprising an instruction for identifying an anomaly when a perturbation extends the cardiac signal beyond an acceptable range in the cardio-profile.

20. The medium recited in claim 18 further comprising instructions for:
preparing a periodic report pertaining to battery charge and operational readiness of the computer system; and
transmitting an alert to a predetermined facility whenever an anomaly requires an active medical response.
